(19) United States
(12) Patent Application Publication
(10) Pub. No.: US 2024/0252276 A1
(43) Pub. Date: Aug. 1, 2024

(54) METHOD FOR PROVIDING AN IMAGE REPRESENTATION BY MEANS OF A SURGICAL MICROSCOPE, AND SURGICAL MICROSCOPE

(71) Applicant: Carl Zeiss Meditec AG, Jena (DE)

(72) Inventors: Felicia WALZ, Oberkochen (DE); Korbinian SAGER, München (DE); Christian LUTZWEILER, München (DE); Lars STOPPE, Jena (DE); Roland GUCKLER, Oberkochen (DE)

(21) Appl. No.: 18/414,998

(22) Filed: Jan. 17, 2024

(30) Foreign Application Priority Data
Jan. 27, 2023 (DE) 10 2023 200 671.5

(51) Int. Cl. A61B 90/00; A61B 90/20; G06V 20/69; H04N 5/265

(52) U.S. Cl. CPC A61B 90/361; A61B 90/20; G06V 20/695; H04N 5/265; A61B 2090/364; A61B 2090/373

(57) ABSTRACT

The invention relates to a method for providing an image representation by means of a surgical microscope, comprising obtaining or capturing at least one application parameter, capturing a color image representation of a capture region by means of a camera, capturing a fluorescence image representation of the capture region by means of a fluorescence camera, processing the fluorescence image representation by means of a processing device to optimize it for an overlay with the color image representation, wherein a type of processing is defined based on the at least one application parameter, overlaying the color image representation with the processed fluorescence image representation, and providing an image signal that encodes the overlaid image representation. The invention further relates to a surgical microscope.

The invention relates to a method for providing an image representation by means of a surgical microscope and to a surgical microscope.

The aim of fluorescence-guided microsurgery is to present additional information to a physician within the operation site. In this case, a fluorescent substance (endogenous or exogenous fluorophore) in the tissue is excited and light emanating from it is captured in a fluorescence image representation. This additional information enables the physician, for example, to compare a blood flow before and after a vascular operation that has been performed and to assess the success of the procedure, or to help distinguish healthy brain tissue from tumor tissue and thereby better protect the healthy tissue in the environment. The decisive factor for these methods is that the fluorescence information is detected as sensitively as possible and presented in detail with good contrast to the surrounding tissue, such that the physician receives extensive additional information about the operation site. In addition, it is important to maintain the white light impression and in particular the structures within the operation site, so that the physician always retains control over or the impression of the condition of the operation site that is independent of the fluorescence information (e.g. the condition of the surrounding vessels during the fluorescence presentation and removal of tumor tissue).

DE 10 2021 203 187 B3 discloses a method for providing an image representation by means of a surgical microscope, comprising: capturing a color image representation of a capture region by means of a camera, capturing a fluorescence image representation of the capture region by means of a fluorescence camera, producing a detailed image from the captured color image representation by means of a spatial filter and an edge stop function, mixing the captured color image representation, the captured fluorescence image representation and the produced detailed image to form a mixed image representation, and providing an image signal which encodes the mixed image representation.

DE 11 2016 004 455 T5 discloses an image processing device, an image processing method, an operating system, and a surgical thread. The image processing apparatus comprises: an image-preserving unit, which receives a first image generated under an illumination condition in which the surgical thread fluoresces and a second image generated under an illumination condition which has at least visible light as images of an operation site using the fluorescent surgical thread, and a synthesis unit which generates a synthetic image obtained by synthesizing a region of the surgical thread of the first image and of the second image.

The invention is based on the object of developing a method for providing an image representation by means of a surgical microscope, and a surgical microscope, in the case of which an overlay of a captured color image representation and a captured fluorescence image representation is improved.

According to the invention, the object is achieved by a method having the features of patent claim 1 and a surgical microscope having the features of patent claim 16. Advantageous configurations of the invention emerge from the dependent claims.

It is one of the basic ideas of the invention to process a captured fluorescence image representation by means of a processing device to optimize it for an overlay with a color image representation. A type of processing is defined here based on at least one obtained or captured application parameter. This allows application-specific control of how an overlaid image representation containing the color image representation and the processed fluorescence image representation is manifested. In other words, for an application-specific scenario, the color image representation can be overlaid with the fluorescence image representation so as to be optimized for that scenario. An image signal encoding the overlaid image representation can thereby better provide a surgeon and/or assistant with the information contained in the capture region.

In particular, a method for providing an image representation by means of a surgical microscope is provided, the method comprising: obtaining or capturing at least one application parameter, capturing a color image representation of a capture region by means of a camera, capturing a fluorescence image representation of the capture region by means of a fluorescence camera, processing the fluorescence image representation by means of a processing device to optimize it for an overlay with the color image representation, wherein a type of processing is defined based on the at least one application parameter, overlaying the color image representation with the processed fluorescence image representation, and providing an image signal that encodes the overlaid image representation.

Furthermore provided is in particular a surgical microscope, comprising a camera configured to capture an image representation of a capture region, a fluorescence camera configured to capture a fluorescence image representation of the capture region, and a processing device, wherein the processing device is configured to process the fluorescence image representation to optimize it for overlay with the color image representation, wherein a type of processing is defined based on at least one obtained or captured application parameter; furthermore to overlay the color image representation with the processed fluorescence image representation, and to provide an image signal which encodes the overlaid image representation.

An advantage of the method and of the surgical microscope is that the image signal for the overlaid image representation can be provided specific to the application. In this way, an optimized or optimal image signal can be provided for each application scenario. The method and the surgical microscope also have the advantage that even in an overlaid or mixed image representation of the captured color image representation and the captured fluorescence image representation, details from the captured fluorescence image representation are retained, so that these can be more easily perceived by a surgeon and/or assistant. In particular, the method and the surgical microscope allow details to be highlighted in the overlaid image representation. In particular, this can also be used to also make, after the overlaying or mixing, vessels, a blood flow and/or tumor structures in the brain clearly visible in the overlaid image representation, while an environment of each of the regions labeled with a fluorescent dye can remain visible. A surgeon and/or assistant can perceive these details more easily as a result and information present in the captured color image representation and in the captured fluorescence image representation can be presented optimally for any application. The still recognizable environment and the still recognizable background can also facilitate orientation within the capture region. For example, in tumor visualization, the overlaid fluorescence signal covers a very large area. By means of the method and the surgical microscope, in particular both a white light impression contained in the color image representation or white light information of the surrounding tissue can be retained, and structures (e.g. vessels etc.) in the background of the fluorescence signal on the tumor can also be presented to be clearly perceivable, so that the structures are not masked too much by the fluorescence signal. An operational sequence and a workflow during surgery can be improved as a result. In addition, errors can be reduced or even avoided.

In particular, an application parameter is a parameter that describes and/or determines an application scenario. Such an application parameter may comprise, for example, information about a fluorescent dye used and/or its physical properties. An application parameter may also comprise information about a user profile, for example about visual impairments of the user (e.g. red-green weakness, etc.) and/or preferences of the user. An application parameter may also comprise information about the planned and/or performed operation or treatment, e.g. an operation type, for example, whether a vascular operation or a tumor resection is performed. An application parameter may also comprise parameters that describe and/or determine (in particular directly or directly) the overlay between the color image representation and the processed fluorescence image representation.

In particular, the processing of the fluorescence image representation is defined based on the at least one application parameter. In particular, a type of processing steps and a processing sequence can be defined based on the at least one application parameter. This can be done, for example, by means of a lookup table in which the manifestations and/or values of the at least one application parameter or combinations thereof are stored, each linked to information describing the type of the processing step(s) and a processing sequence. The processing is then carried out in accordance with the processing steps stored for the at least one application parameter and in accordance with the stored processing sequence. Alternatively or additionally, the type of processing step(s) and the processing sequence can also be defined based on the at least one application parameter by means of an artificial intelligence method, in particular by means of a trained machine learning method, for example by means of a trained neural network. In addition, the color image representation can also be processed taking into account at least one application parameter. In particular, this is implemented by means of the processing device. In particular, a type of processing steps and a processing sequence can similarly also be defined for the color image representation based on the at least one application parameter. The overlaying of the color image representation with the processed fluorescence image representation can also likewise take place taking into account the at least one application parameter. In particular, a type of overlaying steps (addition, fusion, blending, etc.) and, if a plurality of steps are carried out, a processing sequence can also analogously be defined for the overlaying process based on the at least one application parameter. The at least one application parameter can therefore be used to determine a processing of the fluorescence image representation and of the color image representation and also the overlaying and/or to define the steps to be performed in the process and their sequence.

The at least one application parameter can be captured, for example, manually by means of a suitable user interface, for example by means of a display and operating device, wherein the user can define the at least one application parameter (in particular directly or directly) by input and/or selection. Furthermore, the at least one application parameter can also be obtained from a device of the surgical microscope and/or from another (external) device, for example a database system in which planned operations are stored.

In particular, the captured fluorescence image representation is processed by processing picture elements of the fluorescence image representation. In particular, values of the picture elements are selectively modified by means of (digital) image processing. Processing can include one or more of the following methods: brightness compensation, local/global contrast adjustment, gamma correction, amplification, filtering using spatial filters, e.g. Gaussian filters, wavelet filters, bilateral filters, top hat filters, edge filters or frequency filters, noise suppression, global and local histogram equalization (HE) methods, e.g. contrast-limited adaptive histogram equalization (CLAHE), combinations of different processing steps such as noise aware tone mapping (NATM), color channel replacement, morphological processing, conversion into a color space and/or division into a background and foreground, e.g. via segmentation, or Otsu's method. In principle, the processing may also comprise one or more of the following methods: a roll-off correction, a geometric correction (equalization or distortion correction, adjustment of scaling, rotation and/or position for improved overlay), correction of optical aberrations (e.g. chromatic aberrations).

The surgical microscope is in particular a medical surgical microscope, in particular a stereoscopic medical surgical microscope. As a stereoscopic medical surgical microscope, the surgical microscope may in particular comprise a total of two cameras for capturing color image representations (left channel and right channel) and one or two fluorescence cameras. In principle, however, one of the cameras of the stereoscopic medical surgical microscope can also be designed as a fluorescence camera and another camera as a color camera. The surgical microscope in particular comprises a light source for illuminating the capture region in which an object to be captured, in particular a body part of the patient, is arranged, with light, in particular white and/or broadband light. Further, the surgical microscope may also comprise an (additional) excitation light source for exciting the fluorescence dye. The surgical microscope may further comprise optical elements, in particular for focusing and/or magnification purposes. The optical elements may also be part of the camera(s). In particular, the surgical microscope may comprise a lens, which is in particular a common objective in a stereoscopic surgical microscope. Further, a beam splitter may be arranged in a beam path of the surgical microscope in order, for example, to guide light from the capture region to both the camera and the fluorescence camera. Further, optical filters may also be used when capturing fluorescence image representations. The surgical microscope in particular comprises a control device with which the surgical microscope can be controlled. The control device may comprise or provide the processing device. Furthermore, the surgical microscope may comprise a display device and/or a display and operating device. The latter is or are connected to the control device and/or the processing device. Further, the surgical microscope can, as a display device, comprise one or more external display devices, an integrated display device (in particular for the left and the right channel each), a head-up display (HUD) and/or a microdisplay in the optical path.

The camera is in particular designed as a color image detector or white light image detector, that is, the camera is in particular configured to capture light in the wavelength range of white light spatially resolved (in particular two-dimensionally), for example in the wavelength range of visible light. The camera generates and provides in particular a signal which represents an intensity distribution of the light incident on the camera from the capture region, that is, a color image representation (or white light image representation or RGB image representation). In particular, the color image representation is polychromatic. The camera may comprise optical elements (lenses, mirrors, beam splitters, optical filters, etc.) for focusing and/or magnification purposes and/or for beam guidance.

The fluorescence camera is designed in particular as a fluorescence image detector, that is, the fluorescence camera is configured to capture light in the emission wavelength range of at least one fluorescent dye in the capture region spatially resolved. The fluorescence camera generates and provides in particular a signal which represents an intensity distribution of the light incident on the fluorescence camera, that is, a fluorescence image representation. The fluorescence image representation can be monochromatic (e.g. a grayscale image). Basically, the fluorescence imaging representation can also be polychromatic. For example, fluorescence signals from a plurality of emission wavelength ranges can be captured with a fluorescence camera (e.g. a fluorescence signal in the visible range and a fluorescence signal in the infrared wavelength range). The fluorescence camera may comprise optical elements (lenses, mirrors, beam splitters, optical filters, etc.) for focusing and/or magnification purposes and/or for beam guidance and/or filtering.

During overlaying or during mixing, monochromatic image information can be converted into a polychromatic color space or a polychromatic color model, for example if a monochromatic image information item is intended to be overlaid or mixed with a polychromatic image information item. Polychromic image information (e.g. a polychromic fluorescence image representation) can also be converted into or mixed in another polychromatic color space by suitable conversion.

The image signal may have both an analog form and a digital form. In particular, the image signal may also be provided in the form of a digital data packet which is saved in a volatile or nonvolatile memory or storage medium and/or which is output via an interface designed for this purpose.

The overlaid or mixed image representation or image signal can be displayed on a display device.

Parts of the surgical microscope, in particular the control device and/or the processing device, can be designed individually or in combination as a combination of hardware and software, for example as program code, which is executed on a computing device, in particular a microcontroller or microprocessor. However, parts can also be designed individually or in combination as an application-specific integrated circuit (ASIC) and/or field-programmable gate array (FPGA) and/or graphics processor (GPU) and/or digital signal processor (DSP). The control device and/or the processing device may in particular comprise at least one computing device and at least one memory. Furthermore, the components of the surgical microscope have suitable interfaces to allow the exchange of signals and/or data with one another, for example via a bus system designed for this purpose.

In one embodiment, the at least one application parameter is at least partially automatically recognized and/or defined and transferred to the processing device. This allows for the processing of the fluorescence image representation taking into account at least one automatically recognized and/or defined application parameter. This can improve workflow by eliminating the need for manual input by a user, such as a surgeon and/or assistant, to the extent that the at least one application parameter is automatically recognized and/or defined. However, the at least one application parameter may also be defined automatically or semi-automatically based on a user input.

For the automated recognition and/or definition, for example, a suitable lookup table may be provided in which user inputs and/or other parameters, for example, state parameters of the surgical microscope and/or an operation plan, etc. are stored, linked to the at least one application parameter. If necessary, the at least one application parameter is then retrieved from the lookup table. The respective combinations of user inputs and/or other parameters can be ascertained, for example, by means of empirical tests. For example, an operation type may be automatically recognized, for example, by captured image representations of the capture region being evaluated and features in the captured image representation being recognized, such as vascular systems and/or structures and/or a tumor, etc. Furthermore, the at least one application parameter can also be defined automatically based on at least one state parameter of the surgical microscope. State parameters of the surgical microscope may relate in particular to settings of the optical system, such as filter selection, irradiance, a spectrum of the illumination source(s) used, focus settings, magnification settings, etc. For example, the respective settings can be stored in a lookup table linked to the at least one application parameter. If necessary, the at least one application parameter is then retrieved from the lookup table. At least one application parameter may also be estimated based on the above parameters by means of an artificial intelligence method. By way of example, a trained machine learning method, for particular a trained neural network, can be used for this purpose. The automated recognition and/or definition can be carried out, for example, by means of a control device of the surgical microscope, the control device being configured for this purpose, and/or by means of the processing device.

In one embodiment, the processing comprises generating a detail image of the fluorescence image representation, wherein the generation of the detail image takes place taking into account the at least one application parameter, and wherein the overlay with the color image representation is carried out based on the generated detail image. This allows details to be extracted from the captured fluorescence image representation and to be specially marked and/or highlighted when the overlay takes place. In particular suitable filters, in particular two-dimensional spatial filters, can be used to generate the detail image. Further, a brightness and/or contrast in the captured fluorescence image representation can also be modified, for example, a contrast can be enhanced to highlight details in the image representation, in particular to highlight regions with large intensity values in the fluorescence image representation compared with smaller intensity values.

In one embodiment, the generation of the detail image comprises recognizing and removing a background in the fluorescence image representation. In particular, a foreground standing apart from the background in the overlaid image representation can be presented better hereby and image information of the color image representation, which would coincide with the background of the fluorescence image representation, can be reproduced better in the overlaid image representation, to be specific without the recognized and removed background of the fluorescence image representation. In addition, removing the background also allows for improved false-color presentation, because since the background can be removed more effectively, it is less prominent in a false-color presentation. For example, the background can be recognized and removed using suitable filters. These can be, for example, smoothing filters, such as a Gaussian filter. A fluorescence image representation smoothed in this way can then be subtracted from the original fluorescence image representation (picture element by picture element). This creates a detail image with a background which has been removed with respect to the original fluorescence image representation.

In a further embodiment, the generation of the detail image comprises recognizing and/or extracting structures in the fluorescence image representation, wherein the structures are recognized and/or extracted taking into account the at least one application parameter. This allows structural features in the captured fluorescence image representation to be selectively highlighted before overlaying the fluorescence image representation with the color image representation. This makes it in particular possible to highlight structures in the overlaid image representation specific to the application, for example, according to the specific type of operation. For this purpose, selected filters (or filter functions), in particular two-dimensional spatial filters, can be selected in particular specific to the application, to be precise taking into account or based on the at least one application parameter. For example, these may be frequency filters and/or edge filters. Morphological filters and/or Frangi filters may also be used. Furthermore, a local threshold value method (or thresholding) can be used to recognize and/or extract structures. For example, blood vessels and blood flow within the blood vessels can be highlighted particularly well with a top hat filter due to their linear structures and clear edges. A tumor, on the other hand, can be highlighted particularly well due to having a larger area and structures extending to the edge, for example by noise suppression via e.g. a median filter with a subsequent edge detection method, e.g. segmentation and/or thresholding. Furthermore, an artificial intelligence method can also be used to recognize and/or extract the structures in the fluorescence image representation. For example, a trained machine learning method, in particular a trained (deep) neural network, for example a convolution network, may be trained to estimate the structures based on the captured fluorescence image representation and output an image representation that comprises only those structures, or alternatively estimate those picture element coordinates, that reproduce the structures. During a training phase, a large number of fluorescence image representations with structures contained therein are supplied for this purpose to the neural network as input data. For each of the fluorescence image representations, an image representation in which only the structures are reproduced or alternatively, an image representation information item describing the picture element regions in which the structures are contained is simultaneously provided. The neural network is trained with the aid of this information item occurring in pairs, in particular as a part of supervised learning. After the training phase, the trained neural network can then estimate and/or mark, based on a fluorescence image representation, the structures contained in the latter.

In one embodiment, the overlaying comprises identifying a picture element set which reproduces the recognized structures, and, corresponding to the identified picture element set, changing at least one color channel in the color image representation. This allows the recognized structures to be highlighted particularly well in the overlaid image representation. The picture element set can be identified, for example, by means of a filter which is in particular adapted specific to the application and to the structures that are intended to be highlighted (e.g. for highlighting vessels or a tumor). Picture elements corresponding to this picture element set are then modified in the color image representation, wherein this is done for at least one color channel. For example, in an RGB image, the green channel of the picture elements corresponding to the picture elements in the picture element set can be changed in the color image representation. For example, the green values can be amplified. In another example, the green values for the picture elements of the picture element set can be defined by the intensity values of the fluorescence image representation. Similarly, the channels in the LAB color space can be replaced.

In one embodiment, the processing comprises post-processing the generated detail image, wherein the overlaying with the color image representation is carried out based on the post-processed detail image. This allows the details, in particular the structures in the detail image, to be highlighted even better. For example, methods for noise suppression can reduce noise in the detail image. Noise suppression (or denoising) is carried out here in particular specific to the application, i.e. taking into account at least one application parameter. Gamma correction can also be performed. Furthermore, alternatively or additionally, a new filtering can be carried out, for example by means of a top-hat filter and/or by means of another filter function, in order to (further) enhance a local contrast.

In one embodiment, the color image representation is processed by means of the processing device to optimize the overlay, wherein a type of processing is defined based on the at least one application parameter. In particular, a type of processing steps and—in the case of a plurality of processing steps—a processing sequence is defined here based on the at least one application parameter. This also allows the color image representation to be prepared and, in particular, optimized for the overlay. In particular, the methods already mentioned in connection with the processing of the fluorescence image representation, such as filters, gamma correction and/or a change in brightness and/or a change in contrast, etc. can be used in this case.

In one embodiment, the processing of the color image representation comprises generating a color detail image of the color image representation, wherein the generation of the color detail image takes place taking into account the at least one application parameter, and wherein the overlay with the fluorescence image representation is carried out based on the generated color detail image. This also allows details in the color image representation to be better highlighted.

In one embodiment, values in the generated detail image or in the post-processed detail image are amplified by means of a specified gain factor prior to an overlay. This allows the detail image or the post-processed detail image to be more prominently highlighted in the overlaid image representation, so that the details in the detail image, in particular recognized and/or extracted structures, are better visible in the overlaid image representation.

In one embodiment, the processing taking into account the at least one application parameter comprises: a smoothing of the fluorescence image representation by means of a filter, a contrast enhancement and/or a brightness compensation at the smoothed fluorescence image representation and a merging of the generated detail image or the post-processed detail image with the fluorescence image representation thus processed, wherein the overlay with the color image representation is carried out based on the merged image representation. This allows for a particularly information-rich overlay that can be captured intuitively.

In one developed embodiment, the merged image representation is post-processed before the overlay taking into account the at least one application parameter. This allows details in the fluorescence image representation to be better highlighted in the overlaid image representation. For example, post-processing can comprise gamma correction to suppress any background still present and to amplify details.

In one embodiment, the processed fluorescence image representation and the color image representation are overlaid by means of alpha-blending and/or channel replacement in a suitable color space. In particular, this can be used to define a proportion of the respective image representations in the overlaid image representation. Blending and/or channel replacement can also take place, in particular, taking into account the at least one application parameter. Channel replacement can also take place using blending. Color spaces that can be used are RGB, LAB, YUV, or HSV, for example.

In one embodiment, a used alpha value and/or another blending parameter is a function of intensity values of the fluorescence image representation and/or of the processed fluorescence image representation and/or of the detail image and/or of the post-processed detail image. This may provide an overlaid image representation that can be captured highly intuitively, with a highlighted fluorescence image representation.

In one embodiment, the processing comprises converting intensity values of the fluorescence image representation or of the processed fluorescence image representation or of the detail image or of the post-processed detail image into color values according to a color map specified based on the at least one application parameter. This allows details in the fluorescence image representation to be made more easily visible. In particular, an intensity corresponding to the density of the fluorescent dye used can be better presented in the fluorescence image representation, since the intensity values are converted into different colors according to the specified color map. The conveying and/or capture of the information contained in the dye density shown to/by the surgeon and/or assistant can be improved in this way. For example, the color map may be a koufonisi color map, which has been optimized to contrast with bloody tissue. In principle, however, other color maps can also be used.

In particular, the color map may be selected and/or defined based on at least one application parameter. In particular, the color map is optimized for a specific application. For example, the color map may be optimized with regard to personal preferences and/or limitations of a user (e.g. personal preferences with regard to colors, color blindness, etc.). Furthermore, the color map may be optimized with regard to a presentation of known methods and/or known (visual) color gradients. The color map can further be optimized to contrast with a background of the color image representation, e.g. to avoid red colors in a bloody operating environment. The color map may also be optimized with regard to an information presentation, e.g. “heat map” presentations of intensity gradients which are known to the user, in particular with adjusted scaling for an intuitive assessment of the presented information, can be provided.

Further features relating to the configuration of the surgical microscope arise from the description of configurations of the method. Here, the advantages of the surgical microscope are respectively the same as in the configurations of the method.

The invention is explained in greater detail below on the basis of preferred exemplary embodiments with reference to the figures. In the figures:

FIG. 1 shows a schematic illustration of one embodiment of the surgical microscope;

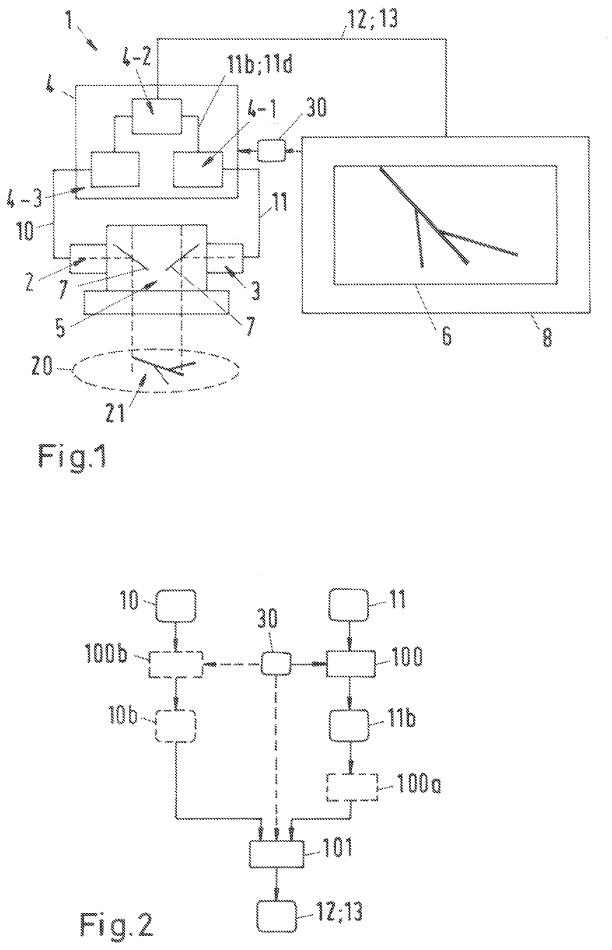

FIG. 2 shows a schematic flowchart for illustrating one embodiment of the method;

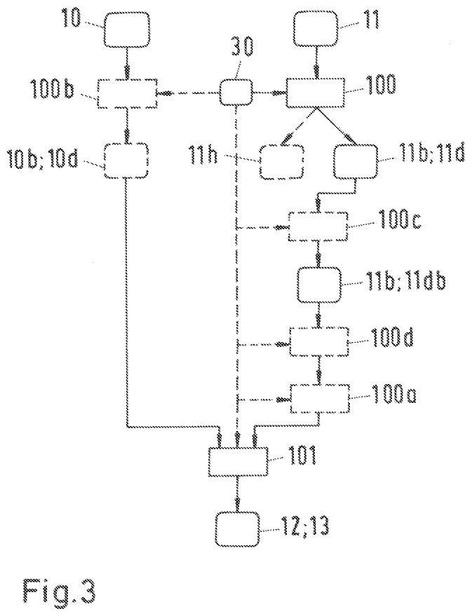

FIG. 3a shows a schematic flowchart for illustrating a further embodiment of the method;

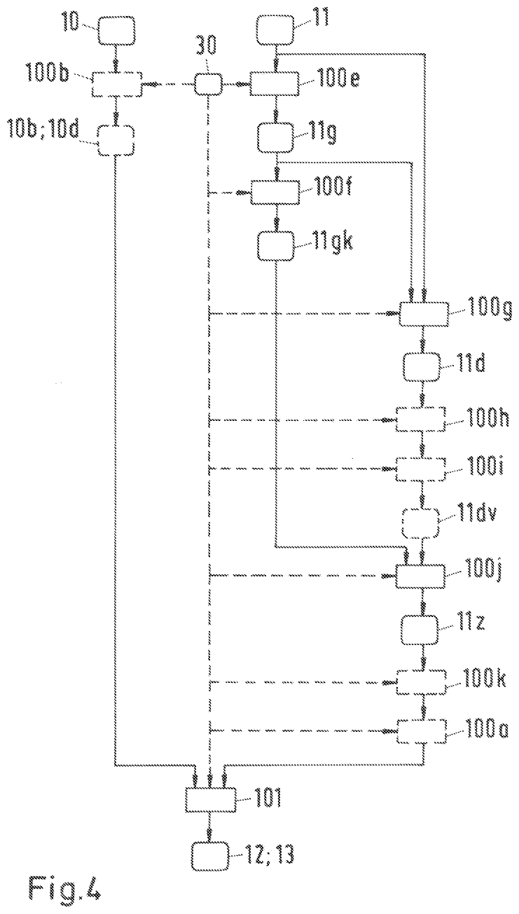

FIG. 4 shows a schematic flowchart for illustrating a further embodiment of the method.

FIG. 1 shows a schematic illustration of one embodiment of the surgical microscope 1. The surgical microscope 1 comprises a camera 2, a fluorescence camera 3, and a processing device 4. The surgical microscope 1 is in particular configured to carry out the method described in this disclosure.

The camera 2 is configured to capture a color image representation 10 of a capture region 20. The fluorescence camera 3 is configured to capture a fluorescence image representation 11 of the capture region 20. The camera 2 and the fluorescence camera 3 capture the capture region 20 for example via a respective beam splitter 7, which is arranged in a beam path of the surgical microscope 1. The camera 2 and the fluorescence camera 3 may be part of a stereo camera system 5 of the surgical microscope 1, which is arranged at a main observer beam path of the surgical microscope 1.

In the example shown, in the capture region 20, an operation scene 21 is arranged in which a fluorescent dye is excited and emits light in a known wavelength range (in particular in near-infrared or infrared), which light is captured by the fluorescence camera 3. At the same time, the operation scene 21 in the capture region 20 is captured as a color image representation 10 in the visible wavelength range by means of the camera 2.

The processing device 4 is configured to process the fluorescence image representation 11 to optimize it for an overlay with the color image representation 10. For this purpose, the processing device 4 comprises a processing module 4-1. The processing module 4-1 comprises, for example, a computing device, for example a microprocessor or digital signal processor, and a memory device, which perform computational operations necessary herefor on the captured fluorescence image representation 11.

A type of processing and, if a plurality of processing steps are provided, in particular also a processing sequence are defined based on at least one obtained or captured application parameter 30. The at least one application parameter 30 can be captured, for example, by means of a display and operating device 8 of the surgical microscope 1, or be obtained and supplied to the processing device 4 in another way. The type of processing to be carried out and the processing sequence can be ascertained, for example, by means of a lookup table provided herefor and/or by means of an artificial intelligence method.

Furthermore, the processing device 4 is configured to overlay the color image representation 10 with the processed fluorescence image representation 11b and to provide an image signal 13 which encodes the overlaid image representation 12. The processing device 4 for this purpose comprises an overlay module 4-2. The overlay module 4-2 comprises, for example, a computing device, for example a microprocessor or digital signal processor, and a memory device, which perform computational operations necessary for the overlay.

The surgical microscope 1 may comprise a display device 6 which may be part of a display and operating device 8. In particular, the overlaid image representation 12 or the provided image signal 13 is output, in particular displayed, on the display device 6. Furthermore, the surgical microscope 1 in particular comprises a control device which comprises, for example, a computing device and a storage device. The control device and the processing device 4 may also be designed as a common device.

The processing device 4 can also be configured to process the color image representation 10 to optimize it for an overlay. The processing device 4 comprises in this respect in particular a further processing module 4-3. The further processing module 4-3 comprises, for example, a computing device, for example, a microprocessor or digital signal processor, and a memory device, which perform computing operations necessary herefor on the captured color image representation 10. A type of processing and, if a plurality of processing steps are provided, in particular also a processing sequence are likewise defined based on the at least one obtained or captured application parameter 30.

FIG. 2 shows a schematic flowchart for illustrating one embodiment of the method. The method is carried out in particular by means of a surgical microscope according to the above-described embodiment.

In a method step 100, the captured fluorescence image representation 11 is processed by means of the processing device, wherein this takes place taking into account the at least one application parameter 30.

In a method step 101, the color image representation 10 and the processed fluorescence image representation 11b are overlaid on or mixed with each other. For this purpose, in a method step 100a, the processed fluorescence image representation 11b, that is, the intensity values, may be converted into a color space (e.g. RGB or LAB). This is done in particular in the color space in which the color image representation 10 is also encoded. In a method step 100b, the captured color image representation 10 may also be processed to optimize the overlay, in particular taking into account the at least one application parameter 30, and the processed color image representation 10b is used in the overlay. Furthermore, the overlay in method step 101 can also be take place taking into account the at least one application parameter 30.

In addition to a direct specification of the at least one application parameter 30 by a user, the at least one application parameter 30 can at least partially automatically be recognized and/or defined and transferred to the processing device 4 (FIG. 1). This can be done, for example, based on user inputs, which can be captured, for example, at a display and operating device 8 (FIG. 1), and/or from state parameters of the surgical microscope 1. For example, a user, for example, a surgeon and/or assistant, can specify a type of operation and/or a type of fluorescent dye, wherein the at least one application parameter is defined and/or estimated from this information, for example using a lookup table and/or an artificial intelligence method. Further, for example, state parameters of the surgical microscope 1 can be read and/or queried, for example in the control device of the surgical microscope 1. In particular a type of the processing steps and a processing sequence of the image representations 10, 11 are selected and/or defined based on the thus recognized and/or defined at least one application parameter 30. This can likewise be done by means of a lookup table and/or by means of an artificial intelligence method.

FIG. 3 shows a schematic flowchart for illustrating a further embodiment of the method. In this embodiment, the processing comprises generating a detail image 11d of the fluorescence image representation 11, wherein the generation of the detail image 11d takes place taking into account the at least one application parameter 30, and wherein the overlay with the color image representation 10 is carried out based on the generated detail image 11d. In particular, the processing device 4 generates in this respect the detail image 11d from the fluorescence image representation 11 in a method step 100.

In particular, the generation of the detail image 11d in method step 100 may comprise recognizing and removing a background 11h in the fluorescence image representation 11.

The generation of the detail image 11d in method step 100 may further comprise recognizing and/or extracting structures in the fluorescence image representation 11, wherein the recognition and/or extraction of the structures take place taking into account the at least one application parameter 30. For example, for recognizing and/or extracting the structures, filtering by means of a filter or a filter function, in particular a two-dimensional spatial filter, for example a morphological filter, a Frangi filter and/or a local threshold method, can be carried out. Alternatively or additionally, the structures can also be recognized and/or extracted by means of an artificial intelligence method.

The overlaying in method step 101 can further comprise identifying a picture element set which reproduces the recognized structures, and, corresponding to the identified picture element set, changing at least one color channel in the color image representation 10.

The processing in a method step 100c may comprise post-processing of the generated detail image 11d, wherein the overlay with the color image representation 10 in method step 101 is carried out based on the post-processed detail image 11db.

The processing of the color image representation 10 in method step 100b may comprise generating a color detail image 10D (processed color image representation 10b) of the color image representation 10, wherein the generation of the color detail image 10d takes place taking into account the at least one application parameter 30, and wherein the overlay with the fluorescence image representation 11 is carried out based on the generated color detail image 10d.

Values in the generated detail image 11d or in the post-processed detail image 11db may be amplified before overlaying in a method step 100d (FIG. 3) by means of a specified gain factor (>1).

FIG. 4 shows a schematic flowchart for illustrating a further embodiment of the method. It is intended that the processing, taking into account at least one application parameter 30, comprises the following procedural steps:
in a method step 100e, a smoothing of the captured fluorescence image representation 11 by means of a filter, for example by means of a bilateral filter or Gaussian filter, so that a smoothed fluorescence image representation 11g is obtained;
in a method step 100f optionally a contrast enhancement and/or a brightness compensation at the smoothed fluorescence image representation 11g, for example by means of the contrast limited adaptive histogram equalization (CLAHE) method or the noise aware tone mapping method, so that a smoothed and corrected fluorescence image representation 11gk is obtained;
in a method step 100g, a detailed image 11d of the fluorescence image representation 11 is obtained by subtracting the smoothed fluorescence image representation 11g from the fluorescence image representation 11; further processing for recognizing and/or extracting structures can be carried out;
in a method step 100j, the generated detail image 11d (or a post-processing detail image 11dv) is merged with the smoothed corrected fluorescence image representation 11gk, wherein the overlay with the color image representation 10 is carried out based on the merged image representation 11z.

In a method step 100h, noise suppression may furthermore be carried out on the detail image 11d. The type of noise suppression is selected, in particular taking into account the at least one application parameter 30.

In a method step 100i, the detail image 11d, in particular intensity values of picture elements of the detail image 11d, can be amplified by means of a gain factor >1, so that an amplified detail image 11dv is obtained. The amplification is carried out in particular so that details, in particular recognized and/or extracted structures, are more prominent in the overlaid image representation 12.

In a method step 100k, the merged image representation 11z can be post-processed before the overlay taking into account the at least one application parameter 30. For example, post-processing can include gamma correction to suppress any background still present and further amplify details.

All method steps (including those of the other embodiments described) are carried out in particular taking into account the at least one application parameter 30, that is, in particular, a type of processing in the method steps and also processing parameters etc., are selected and/or defined based on the at least one application parameter 30. The processing sequence is also selected and/or defined based on the at least one application parameter 30.

It is noted that, depending on the context, the image representations 11d, 11db, 11dv, 11g, 11gk, 11z are comprised of and/or form in particular the term or the reference sign of the processed fluorescence image representation 11b. Furthermore, the term processed color image representation 10b comprises in particular the term color detail image 10d.

The fluorescence image representation 11 and the color image representation 10 may be overlaid by means of alpha-blending and/or channel replacement in a suitable color space. This is done in particular in method step 101 (FIGS. 2, 3 and 4).

A used alpha value and/or another blending parameter can be a function of intensity values of the fluorescence image representation 11 and/or of the processed fluorescence image representation 11b and/or of the detail image 11d and/or of the post-processed detail image 11db.

The processing may comprise converting intensity values of the fluorescence image representation 11 or of the processed fluorescence image representation 11b or of the detail image 11d or of the post-processed detail image 11db into color values according to a color map specified based on the at least one application parameter 30. In the embodiments shown, this can be done, for example, in method step 100a. The color map can, for example, take into account a type of operation and/or the fluorescent dye and/or a user preference based on the at least one application parameter 30. For example, color blindness of the user can be taken into account by means of a suitable color map. Furthermore, visual impressions, which can be perceived by the naked eye, can also be reproduced by means of the color map in the overlaid image representation 12, for example in order to be able to draw on an already existing experience of the surgeon and/or the assistant. Furthermore, signals that are no longer visually perceivable (due to a lack of sensitivity of the eye) or that are assessed very subjectively can also be presented sensitively and objectively in a way that is known to the surgeon from operations in which the signal is sufficiently large. For example, the color map can be a koufonisi color map, which has been optimized to contrast with bloody tissue.

### LIST OF REFERENCE SIGNS

1 Surgical microscope
2 Camera
3 Fluorescence camera
4 Processing device
4-1 Processing module
4-2 Overlay module
4-3 Further processing module
5 Stereo camera system
6 Display device
7 Beam splitter
8 Display and operating device
10 Color image representation
10b Processed color image representation
10d Color detail image
11 Fluorescence image representation
11b Processed fluorescence image representation
11d Detail image
11db Post-processed detail image
11dv Amplified detail image
11g Smoothed fluorescence image representation
11gk Smoothed corrected fluorescence image representation
11h Background
11z Merged image representation
12 Overlaid image representation
13 Image signal
20 Capture region
21 Operation scene
30 Application parameter
100-101 Method steps

1. A method for providing an image representation by means of a surgical microscope, comprising:
obtaining or capturing at least one application parameter,
capturing a color image representation of a capture region by means of a camera,
capturing a fluorescence image representation of the capture region by means of a fluorescence camera,
processing the fluorescence image representation by means of a processing device to optimize it for an overlay with the color image representation, wherein a type of processing is defined based on the at least one application parameter,
overlaying the color image representation with the processed fluorescence image representation, and
providing an image signal that encodes the overlaid image representation,
wherein the processing comprises generating a detail image of the fluorescence image representation,
wherein the generation of the detail image takes place taking into account the at least one application parameter, and
wherein the overlay with the color image representation is carried out starting from the generated detail image.

2. The method as claimed in claim 1, wherein the at least one application parameter is at least partially automatically recognized and/or defined and transferred to the processing device.

3. (canceled)

4. The method as claimed in claim 1, wherein the generation of the detail image comprises recognizing and removing a background in the fluorescence image representation.

5. The method as claimed in claim 1, wherein the generation of the detail image comprises recognizing and/or extracting structures in the fluorescence image representation, wherein the recognition and/or extraction of the structures takes place taking into account the at least one application parameter.

6. The method as claimed in claim 5, wherein the overlaying comprises identifying a picture element set which reproduces the recognized structures, and, corresponding to the identified picture element set, changing at least one color channel in the color image representation.

7. The method as claimed in claim 1, wherein the processing comprises post-processing the generated detail image, wherein the overlaying with the color image representation is carried out based on the post-processed detail image.

8. The method as claimed in claim 1, wherein the color image representation is processed by means of the processing device to optimize the overlay, wherein a type of processing is defined based on the at least one application parameter.

9. The method as claimed in claim 8, wherein the processing of the color image representation comprises generating a color detail image of the color image representation, wherein the generation of the color detail image takes place taking into account the at least one application parameter, and wherein the overlay with the fluorescence image representation is carried out based on the generated color detail image.

10. The method as claimed in claim 1, wherein values in the generated detail image or in the post-processed detail image are amplified by means of a specified gain factor prior to an overlay.

11. The method as claimed in claim 1, wherein the processing taking into account the at least one application parameter comprises: a smoothing of the fluorescence image representation by means of a filter, a contrast enhancement and/or a brightness compensation at the smoothed fluorescence image representation and a merging of the generated detail image or the post-processed detail image with the fluorescence image representation thus processed, wherein the overlay with the color image representation is carried out based on the merged image representation.

12. The method as claimed in claim 11, wherein the merged image representation is post-processed before the overlay taking into account the at least one application parameter.

13. The method as claimed in claim 1, wherein the processed fluorescence image representation and the color image representation are overlaid by means of alpha-blending and/or channel replacement in a suitable color space.

14. The method as claimed in claim 13, wherein a used alpha value and/or another blending parameter is a function of intensity values of the fluorescence image representation and/or of the processed fluorescence image representation and/or of the detail image and/or of the post-processed detail image.

15. The method as claimed in claim 1, wherein the processing comprises converting intensity values of the fluorescence image representation or of the processed fluorescence image representation or of the detail image or of the post-processed detail image into color values according to a color map specified by the at least one application parameter.

16. A surgical microscope comprising:
a camera, which is configured to capture a color image representation of a capture region,
a fluorescence camera which is configured to capture a fluorescence image representation of the capture region,
a processing device, wherein the processing device is configured to process the fluorescence image representation to optimize it for overlay with the color image representation, wherein a type of processing is defined based on at least one obtained or captured application parameter; furthermore to overlay the color image representation with the processed fluorescence image representation, and to provide an image signal which encodes the overlaid image representation,
wherein the processing comprises generating a detail image of the fluorescence image representation,
wherein the generation of the detail image takes place taking into account the at least one obtained or captured application parameter, and
wherein the overlay with the color image representation is carried out starting from the generated detail image.
